(19) United States
(12) Patent Application Publication
(10) Pub. No.: US 2012/0123447 A1
(43) Pub. Date: May 17, 2012

(54) DEVICE FOR TENSIONING FLEXIBLE MATERIAL

(76) Inventors: Ernest Corrao, Bethel, CT (US); Stephen Santangelo, Wethersfield, CT (US); Jeffrey Gelfand, Severna Park, MD (US); Robert S. Collins, Annapolis, MD (US)

(21) Appl. No.: 12/944,379

(22) Filed: Nov. 11, 2010

(51) Int. Cl. A61B 17/04

(52) U.S. Cl. 606/144

(57) ABSTRACT

The present invention provides a device for impart tension to a flexible material, for example, a suture. The device may be used during surgical procedures to ensure the proper tension is exerted.

### FIELD OF THE INVENTION

The present invention relates to a device for imparting tension to a suture. More particularly, the invention relates to an integrated suture tensioning device that creates and maintains suture connection during surgical applications.

### BACKGROUND OF THE INVENTION

It is a common requirement to use devices for tensioning flexible material during surgery to secure tissue, such as a tendon, ligament, or bone under tension to a neighboring tissue (e.g., another bone or bone surface). Suture or other flexible material (i.e., cable, fiber tape, or any other suitable flexible material) can be used to secure tissue (e.g. ligament, tendon, bone) to a nearby bone or other medical device such as a bone plate or expanded washer resting on the surface of a nearby bone. This flexible material will often be tensioned between the neighboring tissue (at one end) and a bone or medical device at the other end. This tension has been accomplished either manually or by a number of devices, such as an adjustment wheel device that has sutures secured to them and are rotated to provide tension to the suture.

Manual tensioning of sutures often results in imprecise tension or a loss of tension during a surgical procedure, for example, as a permanent fixation member is applied. In addition, often such devices require the user to use both of their hands during the tensioning step in a procedure. The use of both hands during a procedure may require an additional person to be present. Further, in some instances it may be difficult to have more than one set of hands involved during a surgical procedure having limited space. This is particularly true when the device needs to create and maintain tension on a suture or flexible material throughout the procedure.

Therefore, there remains a need for a convenient and effective device for creating and then maintaining tension on a suture or flexible material, ideally with one hand. The device would preferably be operable by one individual to securely create and maintain tension on a suture or flexible material while the permanent fixation member is applied.

### SUMMARY OF THE INVENTION

In one embodiment, a device tensioning flexible material (e.g., a suture) of the invention will typically comprise a housing that defines a channel and a shaft slidably disposed in the channel. In one embodiment, the housing may comprise an upper portion, a lower portion, a proximal end and a distal end. The channel may be defined by any portion of the housing, for example, the upper portion of the housing may have a channel extending axially therethrough. A device of the invention may comprise a handle, for example, the lower portion of the housing may comprise a handle. The shaft comprises a proximal end and a distal end and is slidably supported in the housing channel. In some embodiments, the shaft may comprise a plurality of notches that may be disposed along all or a portion of one or more surfaces of the shaft. In some embodiments, the shaft may comprise an engagement member at the proximal end of the shaft.

A device of the invention may comprise a tension anchor. In use, a flexible material to be tensioned may be attached to the tension anchor such that the suture remains fixed with respect to the housing. The tension anchor may be located on any portion of the housing, for example, on a surface of the upper housing. In some embodiments, a tension anchor may comprise a tension carriage member which may slidably engage the upper portion of the housing.

A device of the invention will typically comprise a trigger member comprising an upper portion and a lower portion. In some embodiments, the upper portion includes a drive ratchet comprising a ratchet pawl pivotally mounted thereto. Typically, a spring may be compressibly disposed between the trigger member and the handle for biasing the trigger member and the handle apart. In use, depression of the trigger member causes the ratchet pawl to engage a notch on the shaft and urges the housing toward the distal end of the shaft.

In another aspect, the present invention provides a method of tensioning a suture or flexible material with a tensioning device including positioning the tensioning device such that an engagement member of the device is adjacent to a subject having a suture extending therefrom, attaching the suture to tension anchor and depressing the trigger member to provide tension on the suture. Such methods will typically include maintaining tension on the suture until the suture can be fixed with a tension securing means. Examples of tension securing means include, but are not limited to, a threaded sleeve and screw (e.g., a set screw) construct adapted to engage the suture in a compression fit, a button, or other fixation device known to those skilled in the art.

In another aspect, the present invention provides a method of tensioning a suture or flexible material with a tensioning device in combination with a bone plate, threaded sleeve and a tension securing means. Such methods will typically include positioning the tensioning device such that an engagement member of the device is adjacent to the threaded sleeve through which a suture extends, attaching the suture to the tension anchor and depressing the trigger member to provide tension on the suture. Such methods may also comprise maintaining tension on the suture until the suture can be fixed with a tension securing means and securing the suture with a tension securing means.

In another aspect, the present invention provides a method of tensioning a suture or flexible material with a tensioning device in combination with a washer and a threaded sleeve. Such methods will typically include positioning the tensioning device such that an engagement member of the device is adjacent to the washer and threaded sleeve through which a suture extends, attaching the suture to the tension anchor and depressing the trigger member to provide tension on the suture. Such methods may also comprise maintaining tension on the suture until the suture can be fixed with a tension securing means and securing the suture with a tension securing means.

Other features and advantages of the invention will become apparent to those skilled in the art from the following description of the invention, which refers to the accompanying drawings.

### BRIEF DESCRIPTION OF THE DRAWINGS

FIG. 1 illustrates a perspective view of a device of the present invention.

FIG. 2 illustrates a cross-sectional view of the device.

FIG. 3 illustrates an exploded perspective view of the device.

FIG. 4 illustrates a partial perspective view of an adapter member of the device.

FIG. 5 illustrates the tensioning device of the invention being used to impart tension to a suture that is being used to support a fractured clavicle.

FIG. 6 illustrates a perspective view of the tensioning device of the invention being secured to a threaded sleeve and bone plate.

### DETAILED DESCRIPTION OF THE PREFERRED EMBODIMENTS

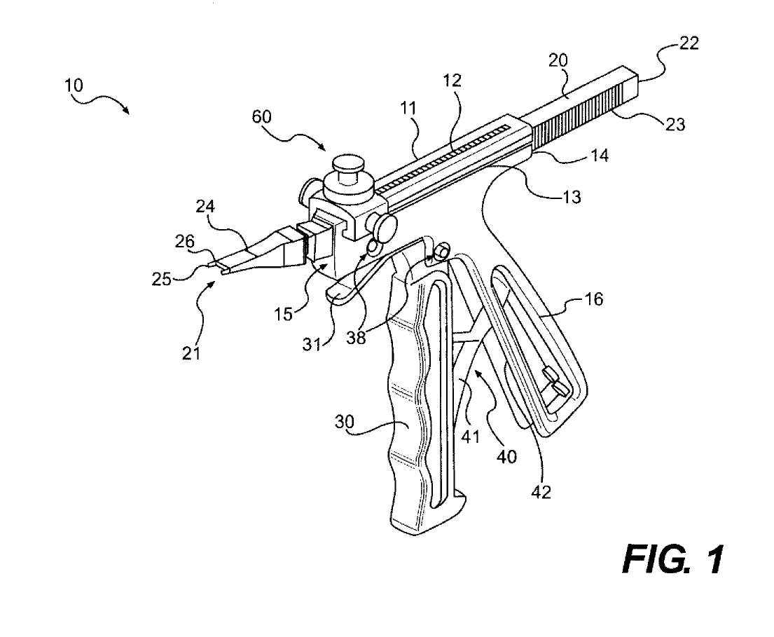

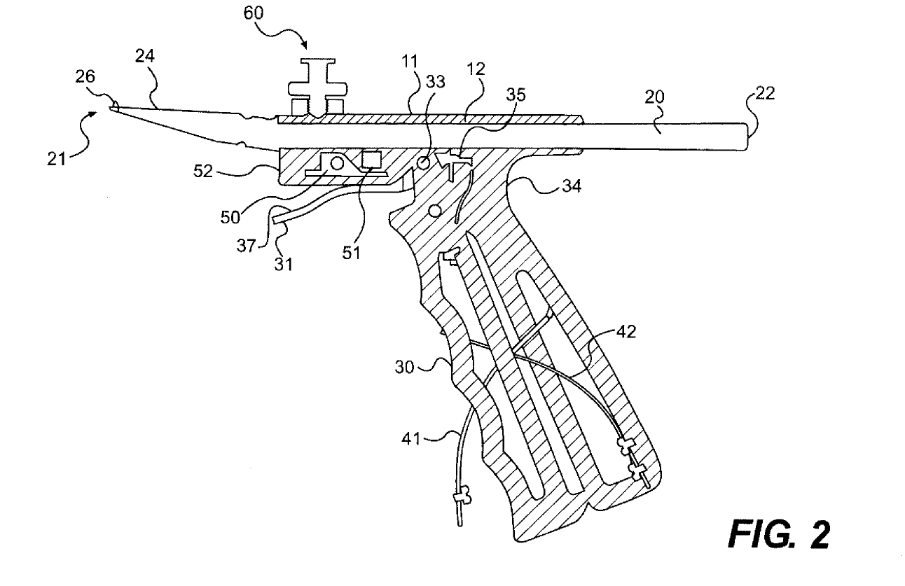

FIGS. 1 to 4 show a device according to an embodiment of the present invention. As shown in FIG. 1, a tensioning device 10 may comprise a housing 11 having an axially extending channel 17 (FIG. 2) formed from a proximal end 15 to a distal end 14. As shown, the housing 11 comprises a handle 16, a lower portion 11b, an upper portion 11a, recessed indentations 12 and a slot 13 extending along a side 18 of the upper portion 11a of the housing 11.

A device of the invention will include a shaft. The shaft may have any cross-section, for example, may have a square cross-section and is disposed in the housing. Other contemplated cross-sections include, but are not limited to, circular, oval, and polygonal (e.g., hexagonal). As shown in the figures, a generally rectangular shaft 20 includes a proximal end 24, a distal end 22 and engagement member 21. The shaft 20 includes a plurality of notches 23 which extend along at least a portion of at least one side of the shaft 20. The engagement member 21 includes engagement fingers 25 and lip 26. The notches 23 may be angled toward the distal end 22 of the shaft 20. The shaft 20 may be rotated prior to use to provide an alternative position of the engagement member 21. The housing 11 is slidably supported on the shaft 20 from the shaft's proximal end 24 to the shaft's distal end 22. The housing 11 is biased in the distal direction of the shaft 20 by a trigger member 30 pivotally connected to the housing 11.

The present invention further contemplates a cannulated shaft. A cannulated shaft may define a bore extending axially therethrough. In some embodiments, the bore of a cannulated shaft may extend from the distal end of the shaft to the proximal end of the shaft. In other embodiments, the bore of the cannualted shaft may extend through a portion of the length of the shaft, for example, from the proximal end or near the proximal end of the shaft to approximately one eight, one fourth, one half or three quarters of the length of the shaft measured from the proximal end. A flexible material (e.g., a suture) may be disposed in the bore of a cannulated shaft.

As shown in FIGS. 1-3, the trigger member 30 includes a drive ratchet 31 pivotally connected to the trigger member 30 at an upper portion 33 that is engageable with a spring 51 biased ratchet pawl 50. The trigger member 30 further includes a spring clip 34 attached to an upper inside surface of the trigger member 30 by a screw 36. The ratchet pawl 50 includes a protrusion 52. The drive ratchet 31 includes a finger compression portion 37 and a hook member 35 and is adapted to be pressed against the ratchet pawl 50 by the spring clip 34 when the trigger member 30 is in a non-compressed position. Hook member 35 is adapted to engage notches 23 of shaft 20 to slide housing 11 at least one notch 23 toward the shaft's 20 distal end. A tension anchor is shown as a tension carriage member 60 may be slidably supported on the slot 13 of the housing 11. The tension carriage member 60 comprises a three-side adjustable post having top post 61 and at least one side post 62. Top post 61 may be attached to the tension carriage member 60 by screw means or any other means contemplated. In the illustrated embodiment, top post 61 is screwed down through an aperture 63 and positioned in at least one of the recessed indentation 12 of housing 11. A two piece spring member 40 comprises a female spring member 41 and a male spring member 42 which connect on at least a portion of one another. The spring member 40 is positioned between the trigger member 30 and handle 16 for biasing the trigger member 30 and handle 15 apart. The male spring member 42 and the female spring member 41 are secured to the trigger member 30 and handle 16 by at least one screw 44.

During operation of the device 10 when the trigger member 30 is depressed the trigger member 30 causes the drive ratchet 31 to engage a notch 23 of shaft 20 with hook member 35 and urges the housing 11 toward the distal end 22 of the shaft 20. In this state, the ratchet pawl 50 is lowered to a point where the protrusion 52 is disengaged from contacting the shaft's notches 23 which permits movement of the housing 11 away from the proximal end of the shaft 20. During operation, a suture (not shown) is secured to a tension anchor, for example, to a post member 61 or 62 of tension carriage member 60 and a pulling force is applied to the suture, thereby providing and maintaining tension to the suture.

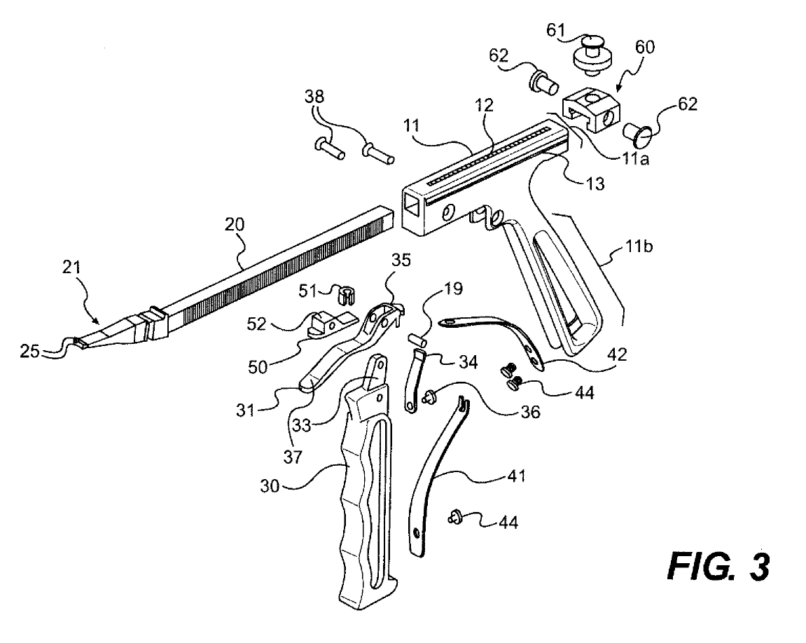

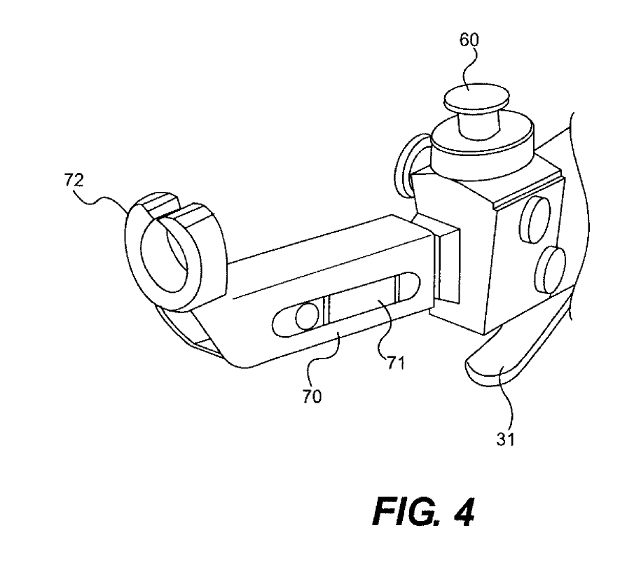

Referring now to FIGS. 3 and 4, device 10 of the present invention provides several advantages, including a capability of creating and maintaining tension on a suture or flexible material. Further, creating and maintaining tension on a suture or flexible material can be accomplished with one hand of a user. The device 10 may further comprise an adapter member 70. The adapter member 70 may comprise a spring member 71 for snap fit connection with the engagement member 21 of the shaft 20. The adapter member 70 may be used to secure connection of the device 10 to a surgical plate, washer or screw. In the illustrated embodiment, the adapter 70 includes an engagement member 72 generally shaped to be fitted to a generally circular member. Engagement member 72 may be provided with a recessed portion into which a washer member may fit. During use the adapter member 70 engages with, for example, a washer member (not shown), which prevents rotation of the washer member while the suture is being tensioned and tension securing means is applied. It being understood that other alternative configurations of engagement members may be used to provide a secured connection.

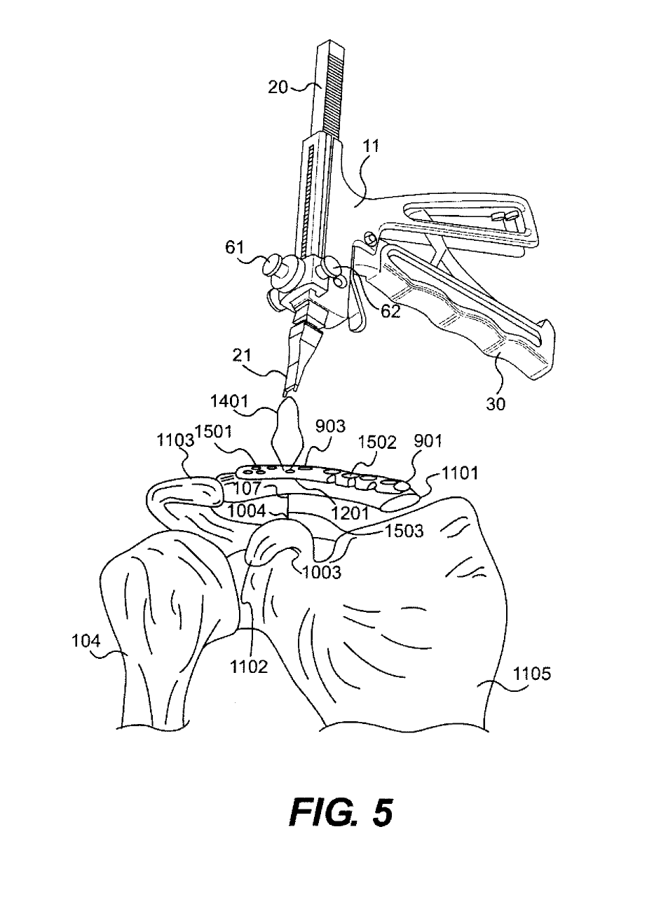

A method of tensioning a suture technique employed in a clavicle fracture repair, for example, is now described with reference to FIG. 5 and according to an embodiment of the present invention. The present invention may be used to secure, however, any type of tissue, for example bone, cartilage, ligament, graft or tendon, such as a biceps tendon, a rotator cuff or a AC separation, which require suture attachment and appropriate tension.

FIG. 5 illustrates a partial perspective view of the bones of the shoulder anatomy illustrating a clavicle 1101 with a fracture 1201 showing the tensioned placement of a bone plate 901 and a suture or other flexible material 1401 undergoing a fracture repair and suture tensioning procedure according to the present invention. The bone plate 901 has been secured to the superior aspect of the clavicle 1101 with a plurality of bone screws 1501 and 1502 inserted into any of a plurality of openings 902 and 905 in openings 902 and 905 in the bone plate 901 and into the clavicle 1101. As known in the art, the bone plate 901 may be a bone screw or plug, for example, employed with or without the bone plate 901.

According to a preferred method of tensioning a suture of the present invention, a length of flexible material 1401 is employed in connection with the bone plate 901 and is placed through an opening 903 of the bone plate 901 through a pre-drilled hole along the fracture 1201 and secured through, around another bone or with an anchor in the bone, for example, a coracoid process 1102. The engagement member 21 of the shaft 20 is positioned in the bone plate opening 903 and a length of flexible material 1401 is secured around tension carriage's post 61 or 62.

For the purposes of illustration and without limitation, the term “suture” or “flexible material” as used herein may be a cable, filament, thread, wire, fiber tape or any other flexible member suitable for bone fixation in the body.

After securing the flexible material 1401 around the through the post 61 or 62 of the tension carriage 60, the user grasps the trigger 30 with one hand, for example the right hand, and compressing the trigger 30 inward to exert tension on the flexible material 1401. Housing 11 slides upward and away from the bone plate 901. As the housing 11 increases its distance from the bone plate 901, tension in the flexible material is increased. Once the desired amount of tension is achieved a securing means can be placed in the opening 903 to maintain the tension between the coracoid process 1102 and the clavicle 1101. In this manner, the flexible material 1401 is precisely tensioned and positioned at an appropriate distance from the bone sections 1102 and 1101. One preferred example of a securing means is a knotless fixation device as shown in published United States patent application US 20100262185 which is specifically incorporated herein by reference in its entirety.

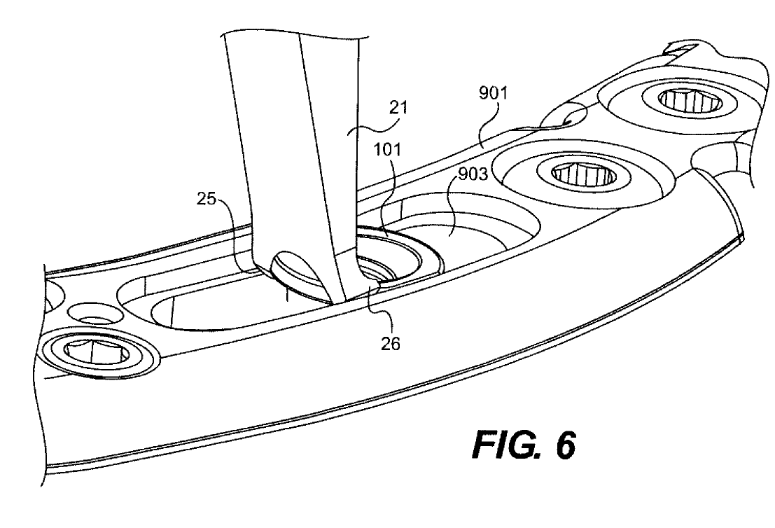

Referring now to FIG. 6, a further embodiment of the device 10 according to the present invention is shown. The engagement member 21 of the shaft 20 is shown in locking engagement with the flange of sleeve 101. During use in this embodiment the engagement fingers 25 are placed against the side of the flange and lip 26 of engagement member 21 is placed on top and around the sleeve's flange to place downward pressure on the sleeve 101. A suture or flexible material may then be extended up through the interior of the sleeve 101 and secured to the device 10 at the tension anchor 60. Once the suture or flexible material is secured to the device 10 the suture can be tensioned according to the aforementioned method and a securing means may be inserted into the sleeve 101 to provide permanent tension to suture. Tension can be maintained on the suture until the securing means firmly engages the sleeve so as to prevent slippage of the suture once the tension is released.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

1. A device for imparting and maintaining tension to a suture comprising:
a housing comprising an upper portion, a lower portion, a proximal end and a distal end, wherein the upper portion has a channel extending axially therethrough and the lower portion comprises a handle;
a shaft having a proximal end and a distal end slidably supported in the housing channel, the shaft comprising a plurality of notches and an engagement member at the proximal end thereof;
a trigger member comprising an upper portion and a lower portion, the upper portion includes a drive ratchet comprising a ratchet pawl pivotally mounted thereto;
a spring compressibly disposed between the trigger member and the handle for biasing the trigger member and the handle apart; and
an adjustable suture post member on the upper portion of the housing;
wherein depression of the trigger member causes the ratchet pawl to engage a notch and urges the housing toward the distal end of the shaft.

2. The device of claim 1, wherein the housing further comprises a top surface and two side surfaces, wherein the top surface includes at least one recessed indentation disposed thereon and the two side surfaces include at least one elongated slot along at one of the two side surfaces.

3. The device of claim 2, wherein the at least one recessed indentation is engaged with a portion of the adjustable suture post member.

4. The device of claim 2, wherein the at least one elongated slot is engaged with a portion of the adjustable suture post member.

5. The device of claim 1, wherein the tensioner further comprises an adapter sleeve for connecting to the proximal end of the shaft.

6. The device of claim 4, wherein the adapter sleeve comprises a retaining spring for maintaining connection with the proximal end of the shaft.

7. The device of claim 1, wherein the shaft has a rectangular shape.

8. The device of claim 7, wherein the shaft has a plurality of notches extending around at least two sides.

9. The device of claim 1, wherein the spring has a female member and a male member, the female member is connected to the trigger member and the male member is connected to the handle.

10. The device of claim 9, wherein the female member is connected to the trigger member by at least one screw.

11. The device of claim 9, wherein the male member is connected to the handle by at least one screw.

12. The device of claim 1, wherein the adjustable suture post member is a three-sided tension carriage, wherein the tension carriage is slidably adjustable on the housing.

13. The device of claim 12, wherein the tension carriage comprises at least one post member threadingly attached.

14. The device of claim 1, wherein the notches are angled toward the distal end of the shaft.

15. The device of claim 1, wherein the engagement member is adapted to be engaged with an adapter member.

16. The device of claim 15, wherein the adapter member comprises a sleeve and a retaining spring.

17. A method of imparting and maintaining tension on a suture, comprising:
positioning a device according to claim 1 such that the engagement member is adjacent to a subject, wherein the subject has a suture extending therefrom;
attaching the suture to the adjustable suture post member;
depressing the trigger member to achieve a desired amount of tension; and
positioning a securing means to the subject for maintaining tension on the suture.

18. The method of claim 17, wherein the device further comprises a top surface and two side surfaces, wherein the top surface includes at least one recessed indentation disposed thereon and the two side surfaces include at least one elongated slot along at one of the two side surfaces.

19. The method of claim 18, wherein the at least one recessed indentation is engaged with a portion of the tension anchor member.

20. The method of claim 18, wherein the at least one elongated slot is engaged with a portion of the adjustable suture post member.

21. The method of claim 17, wherein the device further comprises an adapter sleeve for connecting to the proximal end of the shaft.

22. The method of claim 21, wherein the adapter sleeve comprises a retaining spring for maintaining connection with the proximal end of the shaft.

23. The method of claim 17, wherein the adjustable suture post member is a three-sided tension carriage, wherein the tension carriage is slidably adjustable on the housing.

24. The method of claim 23, wherein the tension carriage comprises at least one post member threadingly attached.
